(19) United States
(12) Patent Application Publication
(10) Pub. No.: US 2016/0278979 A1
(43) Pub. Date: Sep. 29, 2016

(54) OPHTHALMIC TREATMENT DEVICE AND CONTROL METHOD THEREFOR

(71) Applicant: LUTRONIC CORPORATION, Goyang-si, Gyeonggi-do (KR)

(72) Inventor: Gyeong Yeon JEONG, Goyang (KR)

(73) Assignee: Lutronic Corporation, Goyang (KR)

(21) Appl. No.: 14/442,697

(22) PCT Filed: Nov. 13, 2013
(86) PCT No.: PCT/KR2013/010257

§ 371 Date: May 13, 2015

(30) Foreign Application Priority Data
Nov. 13, 2012 (KR) 10-2012-0128071

(51) Int. Cl. A61F 9/008; A61B 3/12

(52) U.S. Cl. CPC A61F 9/008; A61B 3/12; A61F 2009/00897; A61F 2009/00872; A61F 2009/0087; A61B 3/14

(57) ABSTRACT

The present invention relates to an ophthalmic treatment device and a control method improved so as to minimize the treatment time. The ophthalmic treatment device comprise a beam-generating unit for generating a therapeutic beam, a beam-delivery unit for guiding the therapeutic beam to an eyeball, an image unit generating an image of an eyeball to be treated and displaying the image of the eyeball, a pattern unit allowing a predetermined pattern shape to overlie a diseased image area in the image of the eyeball, and an input unit which selects an irradiation position for the therapeutic beam by applying an input signal in such a way as to selectively set a pattern shape overlying either the inside of the diseased portion or the outside of the diseased portion, based on a disease image outline profile.

### BACKGROUND OF THE INVENTION

1. Field of the Invention

The present invention relates to an ophthalmic treating device and a method for controlling the same, and more specifically, to an ophthalmic treating device for treating eye disease and a method for controlling the same.

2. Related Art

An ophthalmic treating device is a device for treating various lesions or diseases caused in eyes. An example ophthalmic treating device radiates therapeutic beams to treat lesions or diseases such as glaucoma, cataract, or macular degeneration. In general, various wavelengths of laser beams may be used for such therapeutic beams.

Such ophthalmic treating device need to diversify the wavelength of laser beams depending on targets under treatment, such as glaucoma in which pressure inside the vitreous body increases, cataract that causes the eye lenses to become white, and macular degeneration that may occur in the retina. The ophthalmic treating device captures and generates images of the eyeball to distinguish between the lesion and the other region in the eyeball. The ophthalmic treating device radiates therapeutic beams to the lesion of the eyeball based on the generated eyeball image.

U.S. Pat. No. 5,549,596B1, titled “SELECTIVE LASER TARGETTING OF PIGMENTED OCULAR CELLS,” as a conventional therapeutic device, discloses a technology in which multiple laser beams are radiated to the patient's lesion in a predetermined area of pattern to thus reduce treatment time.

The conventional therapeutic device illuminates a plurality of lesion spots with therapeutic laser beams in order to decrease time for treatment. However, various shapes of lesions are substantially inconsistent with the constant pattern shapes, and thus, the patterns are formed only on some of the lesion regions, and the other lesion regions need to be arbitrarily radiated with therapeutic laser beams. This may rather prolong the treatment time.

### SUMMARY OF THE INVENTION

The present invention aims to provide an ophthalmic treating device with an enhanced structure and method for controlling the same, which may minimize the time of radiation of therapeutic beams to various shapes of lesions in the eye.

The above objects may be achieved by an ophthalmic treating device having a beam generating unit generating a therapeutic beam and a beam delivering unit guiding the therapeutic beam to an eyeball comprising an imaging unit generating and displaying an image of an eyeball to be treated, a patterning unit allowing a preset pattern shape to overlap an image area of a lesion of the eyeball image, and an input unit applying an input signal to selectively set the pattern shape overlapping one of an inside of the lesion and an outside of the lesion with respect to an outer edge of the lesion image so that a position where the therapeutic beam is to be radiated is set.

Here, the ophthalmic treating device may further comprise a controller controlling the operation of the beam generating unit and the beam delivery unit based on the input signal from the input unit so that the therapeutic beam is radiated to the position of radiation on the lesion image.

Preferably, the controller may control the operation of the beam generating unit and the beam delivery unit so that, if the pattern shape overlapping the outside of the lesion is selectively set with respect to the outer edge of the lesion image by the input unit, the therapeutic beam is radiated to the pattern shape not selected.

Further, preferably, the controller may control the operation of the beam generating unit and the beam delivery unit so that, if the pattern shape overlapping the inside of the lesion is selectively set with respect to the outer edge of the lesion image by the input unit, the therapeutic beam is radiated to the set pattern shape.

The input unit may include a mouse and a digitizer applying the input signal to selectively set the pattern shape.

The patterning unit may include a pattern storing unit storing a plurality of preset pattern shapes and a pattern generating unit enabling any one pattern shape among the plurality of preset pattern shapes stored in the pattern storing unit to overlap the lesion image.

The imaging unit may include an image capturing unit taking an image of the eyeball and generating the eyeball image and the lesion image and a display displaying the lesion image and the eyeball image generated by the image capturing unit and displaying the pattern shape rendered to overlap the image area of the lesion by the pattern storing unit.

On the other hand, the above objects may be achieved by a method for controlling an ophthalmic treating device having a beam generating unit generating a therapeutic beam and a beam delivering unit guiding the therapeutic beam to an eyeball, the method comprising (a) generating an image of an eyeball including a legion, (b) enabling a preset radiation pattern shape to overlap the legion, and (c) selecting an area of the radiation pattern overlapping the lesion to set a position of radiation of the therapeutic beam.

Here, the method may further comprise, if the position of radiation of the therapeutic beam, (d) controlling the beam generating unit and the beam delivery unit so that the therapeutic beam is radiated to the set position of radiation.

Preferably, in step (d), if the pattern shape overlapping the outside of the legion with respect to the outer edge of the legion image, the operation of the beam generating unit and the beam delivery unit may be controlled so that the therapeutic beam is radiated to the pattern shape not selected.

Further, preferably, in step (d), if the pattern shape overlapping the inside of the legion with respect to the outer edge of the legion image, the operation of the beam generating unit and the beam delivery unit may be controlled so that the therapeutic beam is radiated to the set pattern shape.

The ophthalmic treating device may include at least one a mouse and a digitizer applying an input signal to selectively set the pattern shape in step (C).

Other details of embodiments are disclosed in the detailed description and the drawings.

By the ophthalmic treating device and method for controlling the same, according to the present invention, preset pattern shapes are rendered to overlap various shapes of legions, and patterns are selectively set with respect to the outer edges of the legions to set the position of radiation of therapeutic beams, thus minimizing treatment time.

### BRIEF DESCRIPTION OF THE DRAWINGS

FIG. 1 is a view schematically illustrating the configuration of an ophthalmic treating device according to an embodiment of the present invention;

FIG. 2 is a control block diagram illustrating an ophthalmic treating device according to an embodiment of the present invention;

FIG. 3 is a front view illustrating a display of FIG. 1;

FIG. 4 is a view illustrating a radiation pattern stored in the patterning unit;

FIG. 5 is a view schematically illustrating an example of selecting a pattern, with a preset radiation pattern overlapping the lesion of FIG. 3, according to a first embodiment of the present invention;

FIG. 6 is a view schematically illustrating an example of selecting a pattern, with a preset radiation pattern overlapping the lesion of FIG. 3, according to a second embodiment of the present invention; and

FIG. 7 is a flowchart illustrating an example of controlling an ophthalmic treating device according to an embodiment of the present invention.

### DETAILED DESCRIPTION OF THE DRAWINGS

Hereinafter, an ophthalmic treating device and method for controlling the same according to embodiments of the present invention are described in detail with reference to the accompanying drawings.

The ophthalmic treating device is described in a first and second embodiment, wherein the same reference denotations are used to refer to the same elements throughout the specification and the drawings.

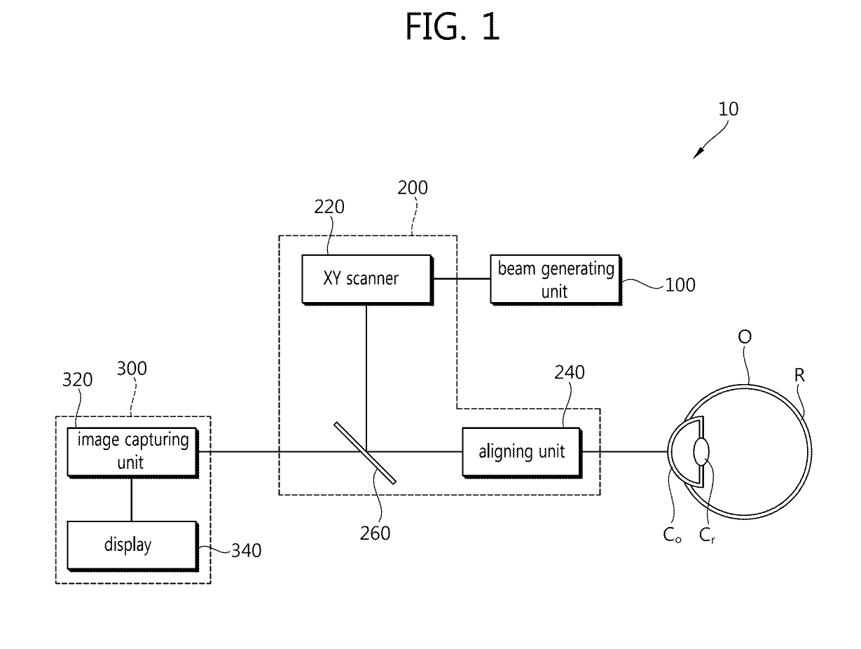

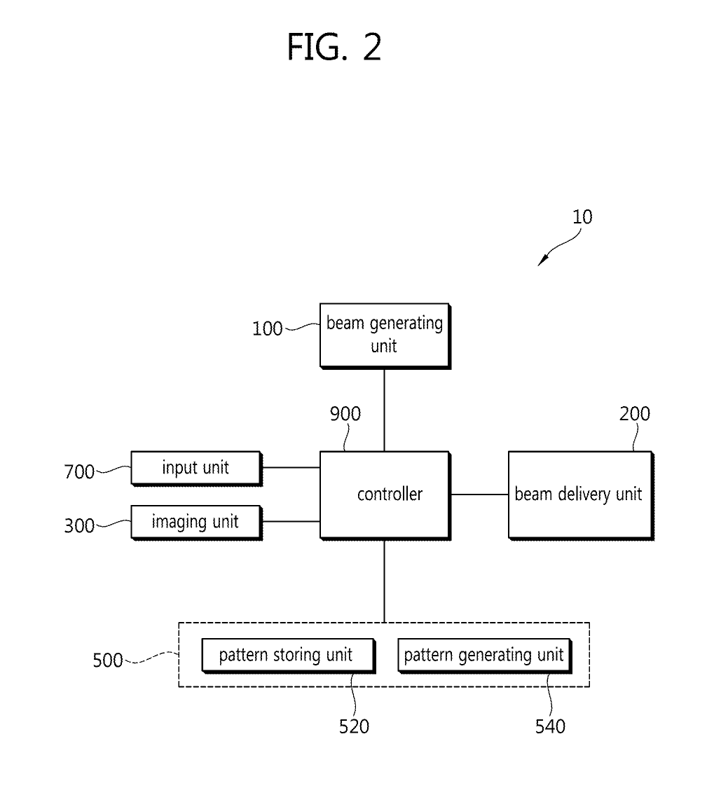

FIG. 1 is a view schematically illustrating the configuration of an ophthalmic treating device according to an embodiment of the present invention. FIG. 2 is a control block diagram illustrating an ophthalmic treating device according to an embodiment of the present invention.

As shown in FIGS. 1 and 2, an ophthalmic treating device 10 according to an embodiment of the present invention includes a beam generating unit 100, a beam delivery unit 200, an imaging unit 300, a patterning unit 500, and an input unit 700. According to an embodiment of the present invention, the ophthalmic treating device 10 is now described as treating the retina R of an eyeball O, but the ophthalmic treating device 10 may also be applicable to the cornea Cr or eye lens Co of the eyeball O.

The beam generating unit 100 is prepared to generate a therapeutic beam. For example, the beam generating unit 100, according to an embodiment of the present invention, may use a switched Nd:YAG. Of course, the beam generating unit 100 using the Q-switched Nd:YAG is a mere example, and this element may adopt other known techniques. The beam generating unit 100 generates a laser beam, as the therapeutic beam. The beam generating unit 100 includes components such as a laser diode (not shown) to radiate the laser beam as the therapeutic beam. Here, the beam generating unit 100 preferably generates a therapeutic beam of a wavelength band depending on the tissue of the eyeball O.

The beam delivery unit 200 includes an XY scanner 220, an aligning unit 240, and a beam splitter 260. The beam delivery unit 200 guides a therapeutic beam radiated from the beam generating unit 100 to the part to be treated, i.e., lesion, of the eyeball O. The above-described beam generating unit 100 and beam delivery unit 200 are operated under the control of a controller 900 to be described below.

The XY scanner 220 is prepared to form a spot of the therapeutic beam on the XY plane positioned in the direction horizontal to the Z axis that is an optical axis of the therapeutic beam radiated to the retina R through the cornea Cr and eye lens Co. The XY scanner 220 includes a plurality of reflecting mirrors (not shown) rotatably provided with respect to different rotational axes. The XY scanner 220 includes at least two reflecting mirrors and may move the spot of the therapeutic beam along the X-axis or Y-axis direction on the XY plane.

The aligning unit 240 is positioned between the eyeball O and the XY scanner 220 and aligns the therapeutic beam incident from the XY scanner 220 to the part to be treated of the eyeball O. The aligning unit 240 includes an object lens consisting of a plurality of lenses. The beam splitter 260 is positioned between the XY scanner 220 and the aligning unit 240 to guide the therapeutic beam from the XY scanner 220 to the aligning unit 240.

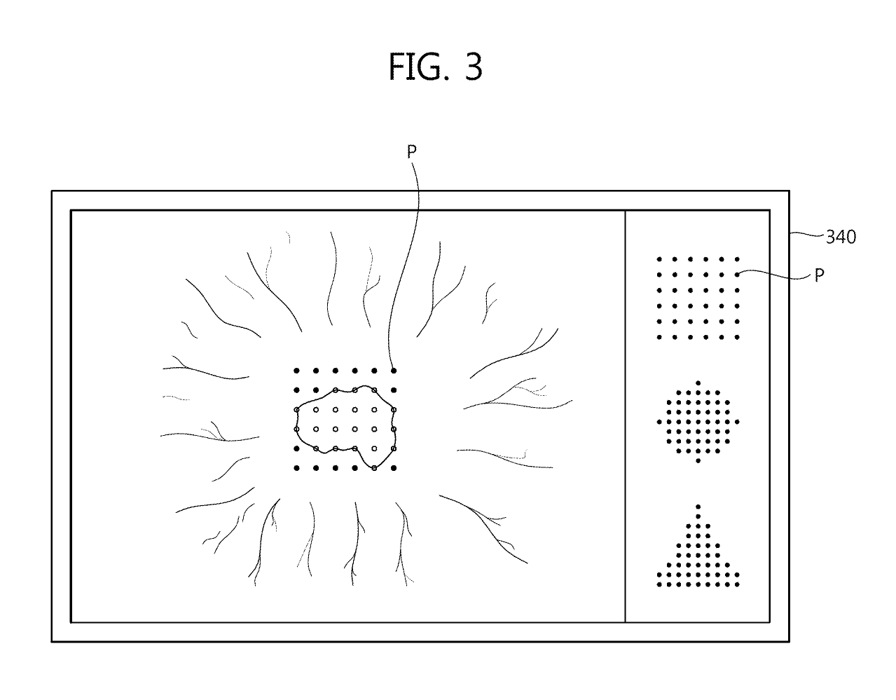

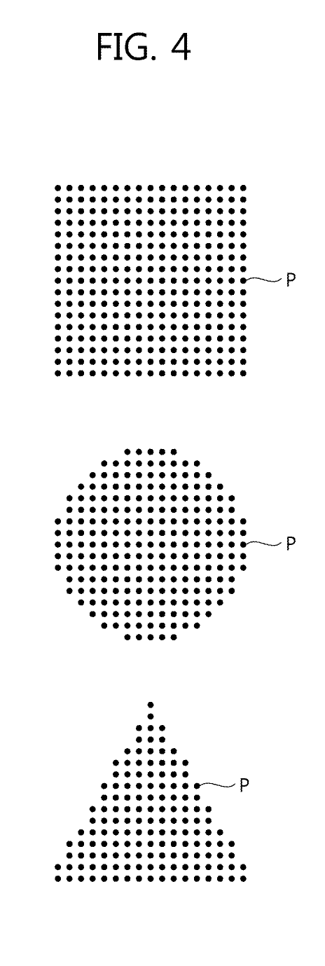

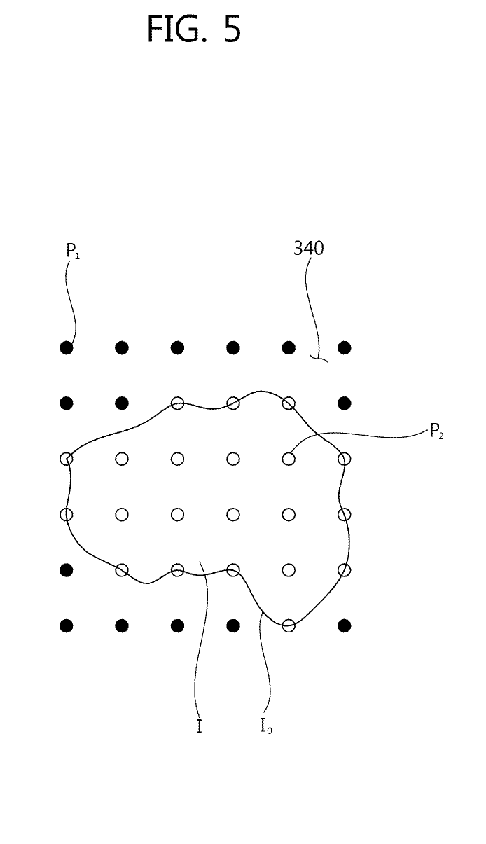

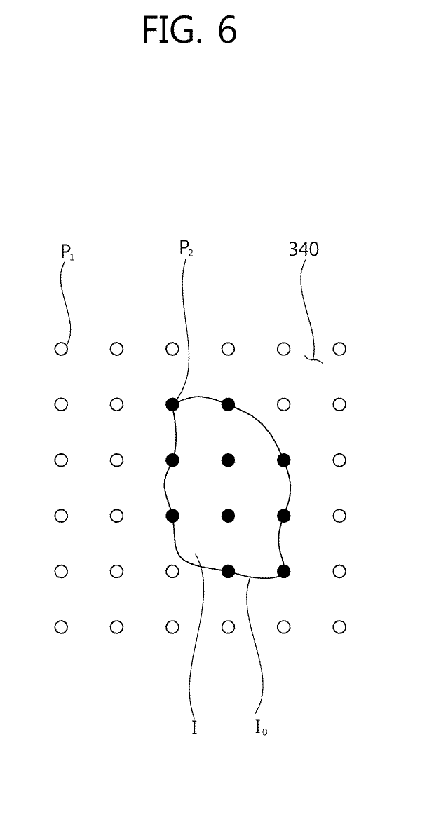

FIG. 3 is a front view illustrating a display of FIG. 1. FIG. 4 is a view illustrating a radiation pattern stored in the patterning unit. FIG. 5 is a view schematically illustrating an example of selecting a pattern, with a preset radiation pattern overlapping the lesion of FIG. 3, according to a first embodiment of the present invention. FIG. 6 is a view schematically illustrating an example of selecting a pattern, with a preset radiation pattern overlapping the lesion of FIG. 3, according to a second embodiment of the present invention. As shown in FIG. 3, the imaging unit 300 generates and displays a fundus image of the eyeball O. Here, the imaging unit 300 generates and displays an image I of the lesion required to be treated, together with the overall fundus image of the eyeball O. The imaging unit 300 includes an image capturing unit 320 and a display 340.

The image capturing unit 320 is used to take a fundus image including the lesion I that has been caused in the fundus, such as, e.g., macular degeneration. The image capturing unit 320 may include at least one imaging device of an optical coherence tomography (OCT) for tomographing the eyeball O and a digital camera.

The display 340 displays the image of the eyeball O, which is taken and generated by the image capturing unit 320, to the user. Specifically, as shown in FIG. 3, the fundus image of the eyeball taken and generated by the image capturing unit 320 may be displayed by the display 340. Here, the fundus image may include the lesion I that is a part to be treated, and allows the operator to identify the part to be treated.

Here, the display 340 may be configured to display a plurality of radiation patterns P pre-stored in the patterning unit 500, described below, so that the operator may select a radiation pattern. Further, the display 340 may be configured so that if the user selects one of the plurality of radiation patterns, the selected radiation pattern may be displayed, selectively overlapping the image I of the lesion.

Meanwhile, the patterning unit 500 is configured to form a pattern overlapping the image I of the lesion generated by the imaging unit 300. According to an embodiment of the present invention, the patterning unit 500 includes a pattern storing unit 520 and a pattern generating unit 540.

The pattern storing unit 520 stores various different pattern shapes in which therapeutic beams may be radiated. As shown in FIG. 4, the radiation patterns P may include a square, a triangle, and a circle, or the radiation patterns P may have an elliptical shape or other various shapes of radiation patterns. Each radiation pattern P includes a plurality of spots where therapeutic beams are radiated. The size of each spot and the intervals between the spots may be configured and adjusted by the user. The radiation patterns may be stored in the pattern storing unit 520 upon manufacture of the product, or may be updated later with additional radiation patterns.

Meanwhile, the pattern generating unit 540 allows a selected one of the plurality of preset patterns stored in the pattern storing unit 520 to overlap the lesion image I.

Specifically, the user may select and set one of the plurality of radiation patterns pre-stored in the pattern storing unit. In this case, the user may select a corresponding radiation pattern similar in shape to the shape of the part to be treated. If the user sets one radiation pattern, the preset radiation pattern may be displayed, overlapping the fundus image displayed on the display as shown in FIG. 3. The user may allow the radiation pattern to overlap the lesion, the part to be treated, by adjusting the position of the radiation pattern displayed on the display.

Here, the pattern shape rendered to overlap the lesion image I by the pattern generating unit 540 is divided into a first pattern P1 and a second pattern P2 with respect to the outer edge Io of the lesion, wherein the first pattern P1 is positioned outside the part to be treated, without overlapping the lesion image I, and the second pattern P2 is positioned inside the outer edge Io, overlapping the lesion image I. The second pattern P2 includes the spots overlapping the outer edge Io of the lesion.

The input unit 700 applies an input signal to selectively set the pattern shape overlapping any one of the inside of the lesion and the outside of the lesion with respect to the outer edge Io of the lesion image I. Based on the input signal selectively set by the input unit 700, the position where the therapeutic beam is to be radiated is set. The input unit 700 may automatically apply the input signal so that any one of the pattern inside the lesion and the pattern outside the lesion with respect to the outer edge Io or the lesion is selected, or the input signal may be manually applied by the operator using at least one a digitizer and a mouse included therein. Here, in case the input unit 700 automatically applies the input signal, the input unit 700 preferably includes a pattern comparing unit (not shown) that compares the number of the pattern spots inside the lesion with the number of the pattern spots outside the lesion with respect to the outer edge Io of the lesion.

The input unit 700 compares the number of pattern spots inside the lesion and the number of pattern spots outside the lesion with respect to the outer edge Io of the selected lesion, i.e., comparison between the number of the spots in the first pattern P1 and the number of the spots in the second pattern P2, and applies an input signal so that a pattern is selectively chosen.

For example, in the first embodiment of the present invention as shown in FIG. 2, the input unit 700, upon determining that the number of the spots in the first pattern P1 overlapping the outside of the lesion is smaller than the number of the spots in the second pattern overlapping the inside of the lesion with respect to the outer edge of the lesion, applies an input signal to select the first pattern. Then, the controller 900 that is to be described below controls the operation of the beam generating unit 100 and the beam delivery unit 200 so that therapeutic beams are radiated to the spots in the second pattern P2 except the selected first pattern P1.

Meanwhile, in the second embodiment of the present invention as shown in FIG. 5, the input unit 700, upon determining that the number of the spots in the first pattern P1 overlapping the outside of the lesion is larger than the number of the spots in the second pattern overlapping the inside of the lesion with respect to the outer edge of the lesion, applies an input signal to select the first pattern. Then, the controller 900 that is to be described below controls the operation of the beam generating unit 100 and the beam delivery unit 200 so that therapeutic beams are radiated to the spots in the selected second pattern P2.

Further, without getting through the above process, the input unit 700 may be configured to directly select, among the radiation patterns, part overlapping the part to be treated, i.e., the pattern with the spots positioned inside the part to be treated and the outer edge of the part to be treated and to radiate a therapeutic beam to the corresponding pattern.

Meanwhile, the outer edge of the part to be treated may be set based on the information directly input by the user (operator) by referencing the fundus image. For example, the user may set the outer edge of the part to be treated by directly drawing the outer edge of the lesion using the above-exemplified digitizer or mouse. Or, the outer edge may be set based on information input by the user through a component provided separately from the input unit.

Or, the outer edge of the part to be treated may be automatically set based on the image information on the fundus image. For example, a part of the fundus image, which has an image parameter, e.g., hue, chroma, or lightness, departing from a normal range, may be selected, and based on the selected part, the part to be treated may be automatically sensed to thus set the outer edge. Or, an outer edge may be computed and recommended to the user, so that the user may easily set the outer edge. In this embodiment, the image capturing unit of the imaging unit or a separate process may be configured to perform such image processing.

Meanwhile, according to an embodiment of the present invention, the ophthalmic treating device 10 further includes a controller 900. The controller 900 controls the operation of the beam generating unit 100 and the beam delivery unit 200 so that a therapeutic beam is radiated to the position of the lesion image I where the therapeutic beam is to be radiated based on an input signal from the input unit 700.

In this case, the controller 900 may store control profiles that enable the optimal control of the beam generating unit 100 and the beam delivery unit 200, corresponding to the plurality of patterns stored in the patterning unit 500. The controller, if one radiation pattern is selected, may reference the control profile corresponding to the radiation pattern, may partially adjust the optimal control profile to exclude the pattern that is excluded by the patterning unit 500 and the input unit 700 among the preset radiation patterns, and may control the operation of the beam generating unit and the beam delivery unit. Specifically, in the first embodiment of the present invention, the controller 900, if the input signal applied by the input unit 700 selects the first pattern P1, controls the operation of the beam generating unit 100 and the beam delivery unit 200 so that the therapeutic beam may be radiated to the second pattern P2 that is not selected. In the second embodiment of the present invention, on the contrary, the controller 900, if the input signal applied by the input unit 700 selects the second pattern P2, controls the operation of the beam generating unit 100 and the beam delivery unit 200 so that the therapeutic beam may be radiated to the selected second pattern P2.

Of course, the controller 900 may differentiate between the first pattern P1 and the second pattern P2 by recognizing the outer edge Io of the lesion so that the above-described control process can be done.

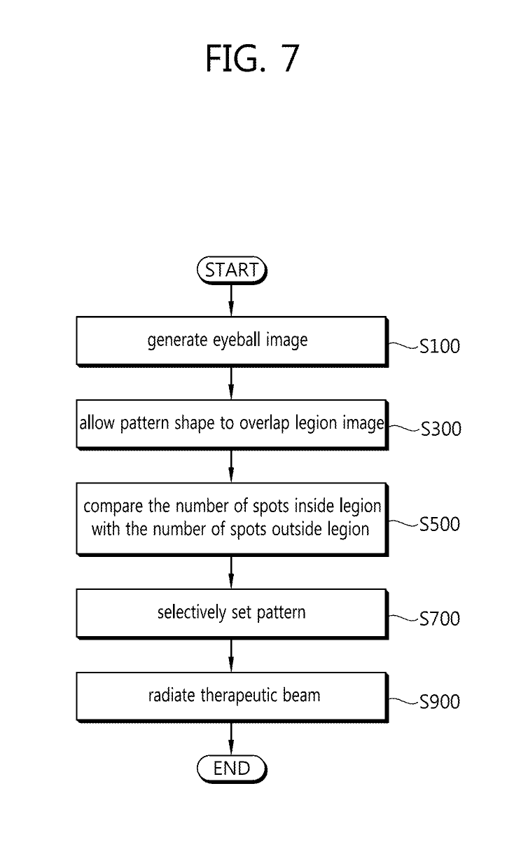

FIG. 7 is a flowchart illustrating an example of controlling an ophthalmic treating device according to an embodiment of the present invention.

Now described is a method for controlling the ophthalmic treating device 10 configured as above, according to an embodiment of the present invention.

First, an image of the eyeball O is generated by the imaging unit 300 (S100). Here, the image of the eyeball O generated at step S100 includes a lesion image I. The patterning unit 500 is used to allow any one of pre-configured pattern shapes to overlap the lesion image I (S300). The pattern shape overlapping the lesion image I is divided into the first pattern P1 and the second pattern P2 with respect to the outer edge Io of the lesion, wherein the first pattern P1 is positioned outside the lesion, and the second pattern P2 is positioned inside the lesion.

The number of spots inside the lesion and the number of spots outside the lesion are compared (S500). That is, the number of spots in the first pattern P1 and the number of spots in the second pattern P2 are compared. One of the first pattern P1 and the second pattern P2 is selected set (S700). Here, an input signal applied from the input unit 700 is applied to select one of the first pattern P1 and the second pattern P2, which has fewer spots than the other.

Lastly, a therapeutic beam is radiated based on the input signal applied from the input unit 700 (S900). In this case, if the first pattern P1 is selected as in the first embodiment of the present invention, the controller 900 controls the operation of the beam generating unit 100 and the beam delivery unit 200 so that the therapeutic beam is radiated to the second pattern P2 that is not selected, while if the second pattern P2 is selected as in the second embodiment of the present invention, the controller 900 controls the operation of the beam generating unit 100 and the beam delivery unit 200 so that the therapeutic beam is radiated to the second pattern P2.

As such, according to the present invention, among a plurality of pre-stored radiation patterns, a radiation pattern similar in shape to the shape of the part to be treated is used, and the pattern setting for radiating the therapeutic beam may be made so that the part of pattern (spots outside the outer edge), which is positioned outside the part to be treated is excluded. According to the conventional art, radiation patterns should be separately designed corresponding to parts to be treated which are different per patient, which is quite time-consuming. According to the present invention, pre-stored radiation patterns are used to minimize the time required for pattern design, and correction is made to exclude where does not overlap the part to be treated among the pre-stored radiation pattern, thus preventing damage to an area other than the part to be treated, which may occur when the therapeutic beam is radiated to the area.

Further, the controller stores the optimal control profiles related to the control of the beam delivery unit, corresponding to the pre-stored radiation patterns. The controller may control the beam delivery based on the control profile to operate, with only the partial pattern not overlapping the part to be treated excluded. According to the conventional part, radiation patterns should be newly designed to each procedure, and it is difficult for the controller to control the beam delivery unit on each pattern in the optimized manner, thus causing delay in light radiation time. However, according to the present invention, the optimized control is possible to minimize the treatment time.

The embodiments in which a separate imaging unit to display an image of the fundus have been described. However, the present invention is not limited thereto, and without providing a separate imaging unit, the conventional slit lamp may be used to configure the ophthalmic treating device. In such case, the user may identify the fundus image using light reflected from the fundus through the ocular lens of the slit lamp. The radiation pattern of the patterning unit may be configured to be identified together with the fundus image by the user through the ocular lens. For example, a separate optic system may be included to radiate a patterned beam to the part to be treated corresponding to the radiation pattern set by the user, so that the radiation pattern overlaps the part to be treated. Or, a separate head-up display may be provided allowing the radiation pattern set by the user onto the light path along which the light is reflected from the fundus to the user, so that the user identifies the image overlapped with the radiation pattern on the fundus.

While the inventive concept has been shown and described with reference to exemplary embodiments thereof, it will be apparent to those of ordinary skill in the art that various changes in form and detail may be made thereto without departing from the spirit and scope of the inventive concept as defined by the following claims.

What is claimed is:

1. An ophthalmic treating device, comprising:
a beam generating unit generating a therapeutic beam;
a beam delivery unit delivering the generated therapeutic beam to a part to be treated of fundus;
an imaging unit displaying a fundus image including the part to be treated;
a patterning unit storing a radiation pattern where the therapeutic beam is to be radiated; and
a controller controlling the beam generating unit and the beam delivery unit to radiate the therapeutic beam corresponding to a pattern in an area overlapping the part to be treated among the radiation pattern.

2. The ophthalmic treating device of claim 1, wherein the radiation pattern of the patterning unit includes a first pattern and a second pattern, wherein the first pattern is positioned outside the part to be treated with respect to an outer edge of the part to be treated, and the second pattern is positioned inside the part to be treated with respect to the outer edge, and wherein the therapeutic beam is radiated to the part to be treated corresponding to the second pattern.

3. The ophthalmic treating device of claim 1, wherein the patterning unit stores a plurality of different radiation patterns where the therapeutic beam may be radiated.

4. The ophthalmic treating device of claim 1, wherein the imaging unit may display the radiation pattern of the patterning unit, selectively overlapping an image of the part to be treated.

5. The ophthalmic treating device of claim 2, wherein the outer edge of the part to be treated is set based on information input from a user.

6. The ophthalmic treating device of claim 2, wherein the outer edge of the part to be treated is automatically computed and set based on information on the fundus image.

7. An ophthalmic treating device having a beam generating unit generating a therapeutic beam and a beam delivering unit guiding the therapeutic beam to an eyeball, the ophthalmic treating device comprising:
an imaging unit generating an image of an eyeball to be treated and displaying the image of the eyeball;
a patterning unit allowing a preset pattern shape to overlap an image area of a lesion image in the image of the eyeball; and
an input unit applying an input signal to selectively set the pattern shape overlapping one of an inside of the lesion and an outside of the lesion with respect to an outer edge of the lesion image so that a position where the therapeutic beam is to be radiated is set.

8. The ophthalmic treating device of claim 7, further comprising a controller controlling the operation of the beam generating unit and the beam delivery unit based on the input signal from the input unit so that the therapeutic beam is radiated to the position of radiation on the lesion image.

9. The ophthalmic treating device of claim 8, wherein the controller controls the operation of the beam generating unit and the beam delivery unit so that, if the pattern shape overlapping the outside of the lesion is selectively set with respect to the outer edge of the lesion image by the input unit, the therapeutic beam is radiated to the pattern shape not selected.

10. The ophthalmic treating device of claim 8, wherein the controller controls the operation of the beam generating unit and the beam delivery unit so that, if the pattern shape overlapping the inside of the lesion is selectively set with respect to the outer edge of the lesion image by the input unit, the therapeutic beam is radiated to the set pattern shape.

11. The ophthalmic treating device of claim 7, wherein the input unit includes a mouse and a digitizer applying the input signal to selectively set the pattern shape.

12. The ophthalmic treating device of claim 8, wherein the patterning unit includes:
a pattern storing unit storing a plurality of preset pattern shapes; and
a pattern generating unit enabling any one pattern shape among the plurality of preset pattern shapes stored in the pattern storing unit to overlap the lesion image.

13. The ophthalmic treating device of claim 8, wherein the imaging unit includes:
an image capturing unit taking an image of the eyeball and generating the eyeball image and the lesion image; and
a display displaying the lesion image and the eyeball image generated by the image capturing unit and displaying the pattern shape rendered to overlap the image area of the lesion by the pattern storing unit.

14. A method for controlling an ophthalmic treating device having a beam generating unit generating a therapeutic beam and a beam delivering unit guiding the therapeutic beam to an eyeball, the method comprising:
(a) generating an image of an eyeball including a legion area;
(b) enabling a preset radiation pattern shape to overlap the legion area; and
(c) selecting an area of the radiation pattern overlapping the lesion to set a position of radiation of the therapeutic beam.

15. The method of claim 14, further comprising, if the position of radiation of the therapeutic beam, (d) controlling the beam generating unit and the beam delivery unit so that the therapeutic beam is radiated to the set position of radiation.

16. The method of claim 15, wherein step (C) is setting the position of radiation of the therapeutic beam excluding a part of the set radiation pattern, which is positioned outside an outer edge of the lesion area.

17. The method of claim 15, further comprising setting an outer edge of the lesion area, wherein the outer edge is set based on information input by a user.

18. The method of claim 15, further comprising setting an outer edge of the lesion area, wherein the outer edge is automatically computed and set based on information on the eyeball image including the legion area.

19. The method of claim 14, wherein the ophthalmic treating device includes at least one a mouse and a digitizer applying an input signal to selectively set the pattern shape in step (C).
